(19) United States
(12) Patent Application Publication
(10) Pub. No.: US 2015/0018945 A1
(43) Pub. Date: Jan. 15, 2015

(54) Small Incision, Customizable Specific Gravity Prosthesis

(71) Applicants: Henry Oksman, Scarsdale, NY (US); Jonathan Fensterheim, New Rochelle, NY (US)

(72) Inventors: Henry Oksman, Scarsdale, NY (US); Jonathan Fensterheim, New Rochelle, NY (US)

(21) Appl. No.: 13/938,536

(22) Filed: Jul. 10, 2013

(51) Int. Cl. A61F 2/02; A61F 2/12

(52) U.S. Cl. CPC A61F 2/02; A61F 2/12
USPC 623/8

(57) ABSTRACT

The present invention relates to a customizable prosthesis, for instance a breast prosthesis, comprised of an outer shell, removable micro-compartments and flexible filling tubes formulated out of soft Poly (methyl 2-methylpropenoate) gel, wherein the removable micro-compartments of the prosthesis are arranged in a hierarchical layering scheme to create a prosthesis capable of undergoing modifications quickly, easily and with minimal invasiveness, while retaining a prosthesis with low specific gravity.

### CROSS-REFERENCE TO RELATED APPLICATION

Not applicable.

### FIELD OF THE INVENTION

The field of the invention relates to medical prosthesis and, more particularly, to those medical prostheses used for breast, testicular, lips or buttocks.

### BACKGROUND OF THE INVENTION

Over the years, various implants have been designed to resemble the feel of a natural breast, or the shape and size of a natural breast. Breast augmentation surgeries have been performed in accordance with a plurality of procedures for decades by medical professionals in this field. In general, these procedures have consisted of inserting a singular sac into each breast and then volumizing the cavity with “fillers” such as silicone, saline, aqueous sugar solutions, aqueous solutions consisting of polyethylene glycol and other chemically inert, non-inflammatory, non-allergenic, and non-carcinogenic compositions.

Silicone fillers in general provide more viscous characteristics and thus more closely resemble the look and feel of a natural breast. However, silicone compounds remain slightly reactive when implanted into one's body and as a result threaten the vitality of the body in the event the silicone implant leaks or ruptures. Under such circumstances, the silicone is capable of moving freely throughout the body and collecting in major bodily organs. The presence of free silicone in the body has incited autoimmune responses in many patients ultimately resulting in the patient's body reaching a severe debilitated state.

Saline implants have provided additional flexibility to the size and symmetry of the breast (e.g., the amount of fluid injected will affect how large the implant or how small the implant would be). However, flexibility to change the size of the breast with time is not simple in these conventional implants and replacement of the implant may be necessary if a change is desired. Furthermore, the weight of the implant becomes an issue when electing to go with a larger implant. The additional weight resulting from the implantation of the larger implant presents an undue burden on the recipient and ultimately may cause ailments in the user (i.e., increased back pain resulting from the weight of a larger breast implant).

### SUMMARY OF THE INVENTION

An implant comprises a biologically proven inert material that encases compartments of fluid and/or gas to form a prosthesis of varied characteristics. The prosthesis contains at least one micro-compartment in a hierarchical layered formation each of which contain filler consisting of liquid, air, gas, gel or combination thereof. The amount of filler injected into each micro-compartment varies depending on the size and shape that the receiving individual seeks to achieve. Moreover, the removable nature of the micro-compartments facilitate one's ability to customize the prosthesis (i.e., if a smaller prosthesis is warranted, a medical examiner may extract filler from the prosthesis using a medical instrument, or even remove the micro-compartments to change the specific gravity of the prosthesis). Modifying the number of micro-compartments and amount of filler in the micro-compartments helps the receiving individual achieve an implant custom to their specific needs.

The customizable prosthesis comprises a flexible outer shell made of soft Poly(methyl 2-methlypropenoate) gel (hereinafter “PMMA”) having at least one flexible filler tube for facilitating the traversal of injected fluids. Within the cavity of the outer shell, there is at least one micro-compartment arranged in a hierarchical layered scheme. The micro-compartments may be filled with fluids and inert gases or combination thereof. After the prosthesis has been placed into the surgically prepared implant site, the filler tube or tubes are drawn to a point that terminates adjacent to the arm pit. The input ports, through which fillers are injected, may be buried under the skin in the vicinity of the armpit at the end of the procedure. These input ports are accessible in the future for the purpose of modifying the structural characteristics and qualities of the prosthesis. For example, as the individual gets older, they may want to modify the size and shape of breast implant, therefore a lighter breast may be achieved by injecting multiple micro-compartment layers comprised of inert-gases behind the first micro-compartment, which can be comprised of a fluid-like solution. Injecting multiple micro-compartments with an inert gas creates an implant that possesses a lower specific gravity than an implant made of purely fluid fillers. As stated above, the injected fillers can be made of saline, silicone gel, PMMA gel, or other fluids and/or gases that are biologically tolerated (e.g., chemically inert compositions). Moreover, the injected filler may further comprise a fluorescent agent to serve as a visual aide for medical professionals when examining the implant in the dark under blue light (e.g., Ultra Violet Light).

The fluorescent agent creates an incentive in the receiving individual to periodically follow up with their physician for check-ups because the examining physician will be able to determine whether the implant has shifted from the implant site or whether any leaks are present when viewed under blue light. The fluorescent agent found in the filler makes it easier to for a medical professional to examine the structure of the prosthesis as a whole with minimal invasiveness. Moreover, at any time after the surgery, the size, shape and/or consistency of the prosthesis may be altered via a simple procedure conducted at an office visit. A medical professional can make a small incision after locating the filler tube under blue light and simply inflate or deflate the prosthesis with a filler until the desired shape, size, weight and/or consistency of the individual is achieved.

The soft PMMA materials have been shown to be biologically inert and have been proven to lack any adverse reactions within the body. PMMA has been a well respected and useful compound for those in the field of Ophthalmology. The physical properties (e.g., durable, light weight, and memory-like qualities) and chemical properties (e.g., inert compound) of PMMA have contributed to the ongoing use by medical professionals today. Today, the soft PMMA materials can be manufactured to conform to the various shapes and firmness values desired by the customer. The design of multiple micro-compartments implanted within the cavity of the outer shell of the prosthesis serves a novel feature in decreasing the total weight of the prosthesis, independent of its size, while simultaneously preserving the qualities of a natural breast. Controlling the specific gravity of the prosthesis is a major breakthrough in the field of cosmetic surgery because the recipient may modify the prosthesis as they get older. For example, as women age the glandular tissues of the breast start to breakdown, ultimately accounting for the cause to ptosis of the breasts. Other relevant factors leading to ptosis of the breasts include a women's body mass index (hereinafter “BMI”), number of pregnancies, whether they have ever breast fed, family history and overall weight. However, the apparatus and method of implementing a prosthesis with customizable features resolves the ptosis of the breasts dilemma. An individual with the implant may at anytime decrease the amount of aqueous filler in the first micro-compartment layer and increase the number of micro-compartment layers filled with inert gases as a means to decrease the specific gravity of the breast. Light weight and more supportive breasts are derived from decreasing the specific gravity of the prosthesis. As such, the present invention facilitates a user's ability to reduce their breast size, increase their breast size and “lift” their breasts via a minimally invasive procedure.

These and other objects, features, and advantages of the present invention will become apparent from the following detailed description of illustrative embodiments thereof, which is to be read in connection with the accompanying drawings.

### BRIEF DESCRIPTION OF THE DRAWINGS

These and other features and advantages of the present invention will be better and more completely understood by referring to the following detailed description of example non-limiting embodiments in conjunction with the drawings, of which:

FIG. 1 illustrates a perspective view of a customizable prosthesis in accordance with one or more embodiments of the present invention.

FIG. 2 illustrates a perspective view of the micro-compartments of a customizable prosthesis in accordance with one or more embodiments of the present invention.

### DETAILED DESCRIPTION

Various aspects and embodiments of the present invention will now be described in detail with reference to the accompanying figures. Certain terminology is used herein for convenience only and is not to be taken as a limitation on the present disclosure. The terminology includes the words specifically mentioned, derivatives thereof and words of similar import. The embodiments illustrated below are not intended to be exhaustive or to limit the disclosure to the precise form disclosed. These embodiments are chosen and described to best explain the principle of the disclosure and its application and practical use and to enable others skilled in the art to best utilize the disclosure.

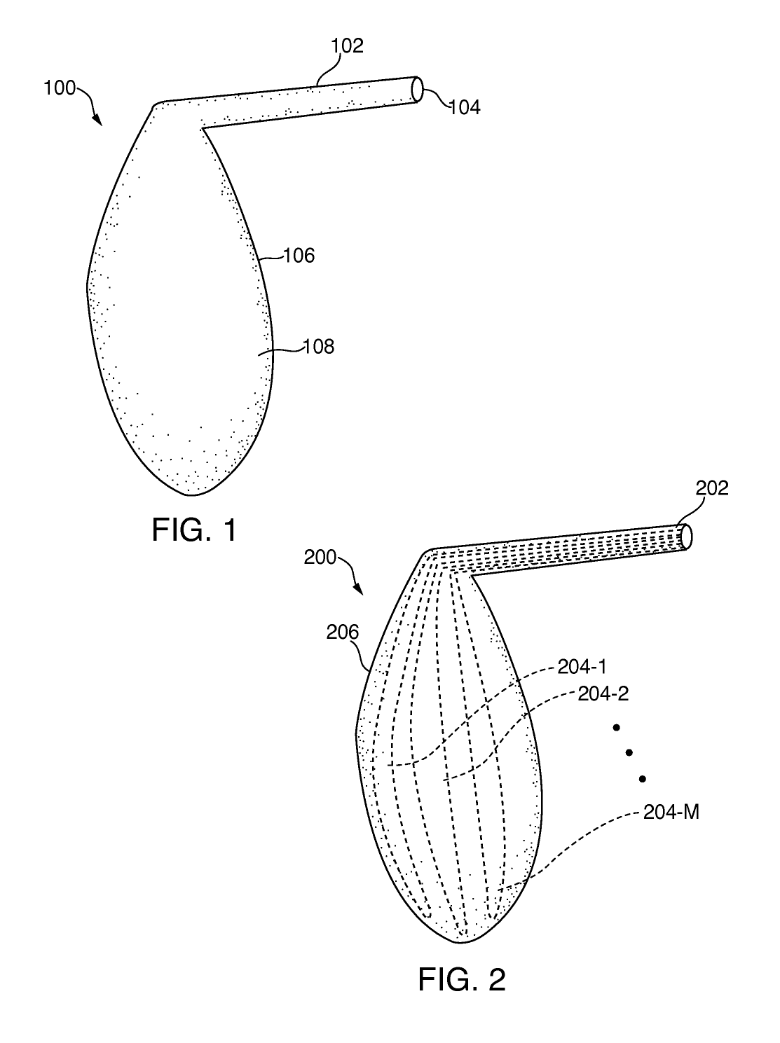

FIG. 1 illustrates a perspective view of a customizable prosthesis 100 designed for surgical implantation into the soft tissue of an individual. Prosthesis 100 is further comprised of flexible implant tube 102, injectable port 104, outer shell 106 and prosthesis cavity 108.

Prosthesis 100 is surgically implanted into an individual at a small localized incision via a medical instrument. Outer shell 106 of prosthesis 100 is formulated out of a polymer (e.g., PMMA) that possesses memory-like and ductile qualities. The structural qualities embraced by outer shell 106 provide the surgeon with several alternative procedures in which to implant the prosthesis (e.g., prosthesis may be implant via a localized incision; prosthesis may be implanted via a medical instrument possessing an injectable means). Upon implantation of prosthesis 100, the volume of prosthesis 100 may be modified in accordance with the patient's specifications. For example, a patient may increase the size of prosthesis 100 via an influx of filler (e.g., saline, PMMA, inert gas, silicone, etc.) injected by a medical instrument at injectable port 104. The filler then traverses along implant tube 102 and empties in prosthesis cavity 108. Alternatively, a patient may decrease the size of prosthesis 100 by retracting filler from prosthesis cavity 108. A single filler or combination thereof may contribute to the volumization of prosthesis 100. Prostheses cavity 108 may consist of a singular inflatable compartment or a plurality of micro-compartments arranged in a hierarchical layered scheme wherein each compartment possesses elastic and memory-like structural characteristics.

Prosthesis 100 is not limited to purely augmentation mammoplasty procedures. An individual may have prosthesis 100 implanted for a plurality of cosmetic procedures including but not limited to lip enhancements, buttock augmentation and testicular implants. The procedures relating to the implant of prosthesis 100 is very similar regardless of the location of the implant itself. A localized incision is made, prosthesis 100 is then implanted at the site of the localized incision, prosthesis 100 is then maneuvered into the proper position, prosthesis fluid is injected by an individual at injectable port 104 via a medical instrument to achieve the desired size, shape and/or firmness and then injectable port 104 is clamped and stowed within the tissue of the individual upon completion. As described above, the filler's injected into prosthesis 100 for augmentation mammoplasty procedures may remain the same for every other cosmetic surgery performed using the described materials.

FIG. 2 illustrates a perspective view of micro-compartments 204 incorporated within customizable prosthesis 200. Micro-compartments 204 are designed to achieve the ideal shape and weight of prosthesis 200 Like outer shell 106, micro-compartments 204 are manufactured from the polymer PMMA. Outer shell 206 is further comprised of at least one flexible implant tube 202 and corresponding injectable port wherein at least one removable micro-compartment 204 extends outward therefrom. Micro-compartments 204 are arranged in a hierarchical layered scheme therefore providing the recipient with a customizable prosthesis. For example, micro-compartment 204-1 represents the first layer of the layering scheme attributed to prosthesis 200. Micro-compartment 204-1 is volumized by an aqueous filler (e.g., saline, PMMA, silicone, aqueous solution, etc.) in order to provide the recipient with the feel and look of a natural breast. Micro-compartments 204-2 . . . 204-10 are then filled with an inert gas to reduce the specific gravity of prosthesis 200 as a whole.

Reducing the specific gravity of prosthesis 200 presents an entirely new novelty in the field of cosmetic surgery. For example, a light weight, natural looking and feeling prosthesis may be implanted into an individual without the unnecessary weight constraints imposed on by today's outdated technology. As described above, the hierarchical layered scheme of micro-compartments 204-1 . . . 204-10 contributes to the phenomena of reducing the specific gravity of prosthesis 200. Similar to the description of prosthesis 100, prosthesis 200 may be modified at any time. For example, a woman who initially received large breast implants may reduce the size of the implant as they get older in order to prevent ptosis of the breast. Implant tube 202 and injectable port 208 is clamped and stowed within the tissue of the individual upon completion. However, a medical professional can make a small incision after locating implant tube 202 under blue light and simply volumize or devolumize prosthesis 200 by injecting a liquid and/or gas until the desired shape, size, weight and/or consistency of the individual is achieved.

An illuminating agent (e.g., fluorescein) is incorporated within implant tube 202 of prosthesis 200. The illuminating agent creates an incentive in the receiving individual to periodically follow up with their physician for check-ups because the examining physician will be able to determine whether prosthesis 200 has shifted from the implant site or whether any leaks are present when viewed under blue light. Furthermore, the illuminating agent makes it easier for a medical professional to examine the structure of prosthesis 200 as a whole with minimal invasiveness. A medical professional can make a small incision after locating filler tube 202 while examining under blue light and simply inflate or deflate micro-compartments 204-1 . . . 204-10 of prosthesis 200 with a liquid and/or gas until the desired shape, size, weight and/or consistency of the individual is achieved.

It should again be emphasized that the above-described embodiments of the invention are presented for purposes of illustration only. Many variations may be made in the particular arrangements shown. For example, although described in the context of particular augmentation mammoplasty procedures, the techniques are applicable to a wide variety of other types of prosthesis implantations. In addition, any simplifying assumptions made above in the course of describing the illustrative embodiments should also be viewed as exemplary rather than as requirements or limitations of the invention. Numerous other alternative embodiments within the scope of the appended claims will be readily apparent to those skilled in the art.

What is claimed is:

1. A prosthesis useful for implanting into soft tissue comprising:
an outer shell;
at least one micro-compartment encapsulated by the outer shell;
an flexible filler tube; and
an injectable port;
wherein the outer shell of the prosthesis further comprises a polymer with memory-like and ductile structural characteristics;
wherein the micro-compartments of the prosthesis are removable and further comprised of a polymer with memory-like and ductile structural characteristics, wherein the micro-compartments are arranged in a hierarchal layering scheme as a means for reducing the overall specific gravity of the prosthesis;
wherein the flexible filler tube functions as a means for traversing prosthesis fillers to the micro-compartments; and
wherein the injectable port functions as a means for modifying the volume of the micro-compartments.

2. The prosthesis of claim 1, wherein the outer shell further comprises at least one flexible implant tube and injectable port wherein at least one removable micro-compartments extends outward there from.

3. The flexible implant tube of claim 2, further comprising a means for opening and closing the injectable port of the implant tube.

4. The means for opening and closing the injectable port of claim 3, further comprising a polymer plug, wherein the polymer plug is formulated to seal the flexible implant tube.

5. The prosthesis of claim 1, wherein the structural composition of the outer shell is comprised of Poly (methyl 2-methylpropenoate).

6. The flexible implant tube of claim 2, wherein the periphery of the tube is sealed by a polymer plug which is then secured to under the patient's tissue by an anchoring means.

7. The hierarchical layering scheme of claim 1 comprising:
a plurality of polymer based micro-compartments arranged in a hierarchal layering scheme;
wherein the first micro-compartment is volumized via an aqueous solution;
wherein the second micro-compartment and proceeding micro-compartment thereafter is volumized via an inert gas.

8. The volumization of the first micro-compartment according to claim 6, wherein the aqueous solution is comprised of soft Poly (methyl 2-methylpropenoate) gel, silicone, saline, glucose based solution, water, polymer, or combination thereof.

9. The second and all proceeding micro-compartments thereafter according to claim 6, further comprising a means for reducing the specific gravity of a prosthesis, wherein the second and all proceeding micro-compartments are volumized by an inert gas.

10. The prosthesis of claim 1, further comprised of an illuminating agent capable of being detected under Ultra Violet light, wherein the illuminating agent facilitates examination of the prosthesis.

11. The illuminating agent of claim 10, wherein the illuminating agent is fluorescein.

12. The prosthesis of claim 1, wherein the soft tissue further comprises breasts.

13. The prosthesis of claim 1, wherein the soft tissue further comprises buttock.

14. The prosthesis of claim 1, wherein the soft tissue further comprises lips.

15. The prosthesis of claim 1, wherein the soft tissue further comprises testicles.

16. The prosthesis of claim 1, wherein the micro-compartments are spherical in shape.

17. A method for reducing the specific gravity of a prosthesis, comprising:
arranging the micro-compartments of the prosthesis in a hierarchical layering scheme;
volumizing at least two micro-compartments; and
sealing the prosthesis;
wherein arranging the micro-compartments in a hierarchical layering scheme is further comprised of designating the first layer to be volumized by an aqueous solution and designating the second and proceeding micro-compartments thereafter to be volumized by an inert gas;
wherein the first layer is volumized by an aqueous solution and the second and all proceeding layers thereafter are volumized by an inert gas;
wherein the sealing of the prosthesis is performed via a pressure cap formulated from soft Poly (methyl 2-methylpropenoate) gel capable of being easily removed to perform modifications to the prosthesis.

18. The method of claim 17, wherein the hierarchical layering scheme serves as a means for reducing the specific gravity of the prosthesis.

19. The method of claim 17, wherein the micro-compartments volumized by injecting the prosthesis filler into the micro-compartments via a medical instrument.

20. The medical instruments of claim 19, wherein the medical instrument is a syringe.
